(19) United States
(12) Patent Application Publication
(10) Pub. No.: US 2002/0002184 A1
(43) Pub. Date: Jan. 3, 2002

(54) Novel process and compound

(75) Inventors: Victor Witold Jacewicz, Tunbridge Wells (GB); Neal Ward, Crowborough (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham plc

(21) Appl. No.: 09/925,859

(22) Filed: Aug. 9, 2001

Related U.S. Application Data
(63) Continuation of application No. 09/342,761, filed on Jun. 29, 1999.

(30) Foreign Application Priority Data
Jan. 15, 1997 (GB) 9700692.8

(51) Int. Cl. A61K 31/4525; C 07D 4 7/02

(52) U.S. Cl. 514/321; 546/197

(57) ABSTRACT

Paroxetine hydrochloride is obtained in a free-flowing and easily soluble form (suitable for preparing solid formulations or aqueous solutions, suitable for parenteral use) by spry-drying solutions of paroxetine hydrochloride hemihydrate or other anhydrate/hydrate/solvate/amorphous forms.

The present invention relates to a process for the preparation of a pharmaceutically active compound, and to use of the so-prepared compound in therapy. In particular this invention is concerned with the preparation of a free-flowing form of paroxetine hydrochloride.

Pharmaceutical products with antidepressant and anti-Parkinson properties are described in U.S. Pat. No. 3,912,743 and U.S. Pat. No. 4,007,196. An especially important compound among those disclosed is paroxetine, the (−)trans isomer of 4-(4′-fluorophenyl)-3′,4′-methylenedioxyphenoxymethyl)-piperidine. This compound is used in therapy as the hydrochloride salt to treat inter alia depression, obsessive compulsive disorder (OCD) and panic.

Paroxetine hydrochloride has been described in the literature as a crystalline hemihydrate (see EP-A-0223403 of Beecham Group) and as various crystalline anhydrate forms (see WO96/24595 of SmithKline Beecham plc). These known forms have properties that are not ideal for all pharmaceutical applications, and are prepared by multi-step procedures involving precipitation under carefully controlled conditions, filtration, drying, and homogenisation. The preferred crystallisation procedures utilise organic solvents which, when compared to water, are costly and are associated with safety and environmental problems. Furthermore, the difficulty of producing crystalline products with a uniform and regular particle size causes problems with formulation by encapsulation. Also, the flow characteristics of crystalline products limit the choice of bulk transfer and formulation technologies that can be used, while dust formation and electrostatic properties can be hazardous. In addition, the known sold forms of paroxetine hydrochloride are relatively insoluble and are slow to dissolve completely.

There remains a need for a form of paroxetine hydrochloride with improved processing and formulation characteristics.

According to a first aspect of the invention, there is provided a process for preparing a free-flowing form of paroxetine hydrochloride which comprises spray drying a solution of paroxetine hydrochloride.

The feedstock for spray drying may be prepared conveniently by, for example, dissolution of paroxetine free base in aqueous hydrochloric acid, although other solid forms of paroxetine hydrochloride may also be dissolved. For example, the feedstock may be prepared by dissolving amorphous paroxetine hydrochloride or a crystalline paroxetine hydrochloride anhydrate, hydrate or solvate in suitable solvent. The solvent used may be pure water or a mixture of water with compatible organic solvents. Suitable compatible organic solvents include pyridinem, acetic acid, acetonitrile, acetone, ethanol, propan-1-ol, butan-1-ol and tetrahydrofuran. Or alternatively a suitable organic solvent may be used on its own to form a solution with paroxetine hydrochloride. Some heating may be used to achieve and maintain complete solution, though once dissolved and in the absence of seeds of a crystalline form, aqueous solutions are stable at ambient temperature for many days. Suitable concentrations of paroxetine hydrochloride for spray-drying are in the range 1 to 30% by weight, preferably in the range 5% to 20% by weight.

Using conventional spray-drying procedures under normal conditions, often results in paroxetine hydrochloride particles that are sticky and adhere to the sides of the apparatus and to each other. However, when apparatus and operating conditions are selected to ensure that the particles are cooled sufficiently before they strike the apparatus walls, successful spray-drying may be carried out. Careful control of drop size in the spray nozzles, air flow rates and temperatures is needed to suit the apparatus used.

The paroxetine product of the above process is free-flowing, is readily wetted, and dissolves rapidly; solutions with high concentrations may be prepared without recourse to heating.

Accordingly, a second aspect of this invention is spray-dried paroxetine hydrochloride.

Spray-dried paroxetine hydrochloride of this invention has been found to be particularly suitable for applications where uniform particle size and good flow properties are advantageous. Furthermore as a result of the close control of particle size possible by spray-drying, the product may be handled conveniently and safely without the hazards associated with the dust produced when conventionally prepared paroxetine hydrochloride solids are prepared. Examples of applications where uniform particle size are advantageous include controlled release and microencapsulation (coated particle technology). Samples may be produced with particle sizes for specific applications, for example in the range 10-1000 microns.

Microencapsulation may be incorporated into the spray-drying process or may be carried out in a subsequent step. This technology is useful for taste masking, rapid or controlled release formulations, hence control of pharmacokinetics including the matching of pharmacokinetic properties for combination products.

Isolation of the solid product from the feedstock solution may be possible with just one processing stage; and so there is generally no need for blending, granulating, or drying, though an extra drying stage may be added if required. Providing aqueous feedstocks are used the costs and environmental problems normally associated with organic solvents are entirely avoided.

The spray-dried product of this invention may be formulated for therapy in the dosage forms described in EP-A-0223403 or WO96/24595. The free-flowing properties are advantageous for the preparation of solid formulations. Also the easily soluble nature of spray dried paroxetine hydrochloride makes it suitable for the preparation of solutions for parenteral use.

Therapeutic uses of the paroxetine product of this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the disorders”.

Accordingly, the present invention also provides:

a pharmaceutical composition for treatment or prophylaxis of the disorders comprising spray-dried paroxetine hydrochloride and a pharmaceutically acceptable carrier or an aqueous solution of reconstituted spray-dried paroxetine hydrochloride;

the use of spray-dried paroxetine hydrochloride to manufacture a medicament in solid or reconstituted liquid form for the treatment or prophylaxis of the disorders; and

a method of treating the disorders which comprises administering an effective or prophylactic amount of spray-dried paroxetine hydrochloride as a solid oral composition or as a reconstituted aqueous oral or parenteral composition to a person suffering from one or more of the disorders.

The invention is illustrated by the following Example.

### EXAMPLE

A 10% aqueous solution of paroxetine hydrochloride is spray-dried under the following conditions:

| | |
|---|---|
| Apparatus: | Niro Fielder Mobile Minor |
| Inlet temperature setting: | 185° C. |
| Actual inlet temperature: | 184-185° C. |
| Outlet temperature: | 94-95° C. |
| Atomiser speed: | 40,000-50,000 rpm |
| Pump speed (peristaltic): | 32-34 rpm |
| Air supply | 4.8-5.2 bar |
| DP across filters: | |
| Bag filter: | start of run 57 mm of water |
| | end of run 65 mm of water |
| Hepa filter: | start of run 7 mm of water |
| | end of run 7 mm of water |
| DP across the orifice plate: | start of run 80+ mm of water |
| | end of run 80+ mm of water |

1. A process for preparing a free-flowing form of paroxetine hydrochloride which comprises spray drying a solution of paroxetine hydrochloride.

2. A process according to claim 1, in which the feedstock for spray drying is prepared by dissolution of paroxetine free base in aqueous hydrochloric acid.

3. A process according to claim 1, in which the feedstock is prepared by dissolving amorphous paroxetine hydrochloride or a crystalline paroxetine hydrochloride anhydrate, hydrate or solvate in a suitable solvent.

4. A process according to claim 1, in which the solvent is pure water or a mixture of water with compatible organic solvents.

5. A process according to claim 1 in which the solution of paroxetine hydrochloride is in a suitable organic solvent optionally mixed with water.

6. A process according to claim 5 in which the organic solvent is selected from pyridine, acetic acid, acetonitrile, acetone, ethanol, propan-1-ol, butan-1-ol, or tetrahydrofuran.

7. Spray-dried paroxetine hydrochloride.

8. A pharmaceutical composition for treatment or prophylaxis of the disorders comprising spray-dried paroxetine hydrochloride and a pharmaceutically acceptable carrier or an aqueous solution of reconstituted spray-dried paroxetine hydrochloride.

9. A method of treating the disorders which comprises administering an effective or prophylactic amount of spray-dried paroxetine hydrochloride as a solid oral composition or as a reconstituted aqueous oral or parenteral composition to a person suffering from one or more of the disorders.
